(19) United States
(12) Patent Application Publication
(10) Pub. No.: US 2013/0138097 A1
(43) Pub. Date: May 30, 2013

(54) SYSTEM AND METHOD TO DETECT PATIENT RETURN ELECTRODE CONNECTION IN AN RF ABLATION SYSTEM

(71) Applicant: MEDTRONIC ABLATION FRONTIERS LLC, Minneapolis, MN (US)

(72) Inventors: Charu P. Mathur, San Diego, CA (US); Marshall L. Sherman, Cardiff by the Sea, CA (US)

(73) Assignee: MEDTRONIC ABLATION FRONTIERS LLC, Minneapolis, MN (US)

(21) Appl. No.: 13/749,742

(22) Filed: Jan. 25, 2013

Related U.S. Application Data
(63) Continuation-in-part of application No. 12/696,910, filed on Jan. 29, 2010.
(60) Provisional application No. 61/635,434, filed on Apr. 19, 2012.

(51) Int. Cl. A61B 18/18; A61B 18/16

(52) U.S. Cl. CPC A61B 18/18; A61B 18/16
USPC 606/33; 606/35

(57) ABSTRACT

A medical device is provided, including an ablation system with a generator, at least one ablation element, a patient return electrode, and a feedback system to verify and monitor the electrical connection between the generator and the patient return electrode, as well as contact of the patient return electrode with the patient. The medical system sends a test signal to the patient return electrode prior to the delivery of ablation energy. If the response to the test signal indicates that the connection of the patient return electrode is absent, intermittent, or of low quality, the medical device may also provide notice, a warning or alarm.

### FIELD OF THE INVENTION

The present invention relates generally to medical systems and methods of use thereof, and more particularly to an ablation system for detecting and monitoring a patient return electrode.

### BACKGROUND OF THE INVENTION

Numerous procedures involving catheters and other minimally invasive devices may be performed for a wide variety of treatments, such as ablation, angioplasty, dilation or other similar therapies. For example, there are many variations of cardiac arrhythmias with different causes, including atrial fibrillation, generally involving irregularities in the transmission of electrical impulses through the heart. To treat cardiac arrhythmias or irregular heartbeats, physicians often employ specialized ablation catheters to gain access to interior regions of a patient's body. Such catheters include tip electrodes or other ablating elements to create ablation lesions that physiologically alter the ablated tissue without removal thereof, disrupting or blocking electrical pathways through the targeted tissue. In the treatment of cardiac arrhythmias, a specific area of cardiac tissue, such as for example atrial rotors, having aberrant electrically conductive pathways with erratic electrical impulses is initially localized. A medical practitioner (such as a physician) may direct a catheter through a body passage including for example a blood vessel into the interior region of the heart that is to be treated. Subsequently, the ablating portion of the selected device is placed near the targeted cardiac tissue to be ablated, such as for example a pulmonary vein ostium or atrium.

An ablation procedure may involve creating one or more lesions in order to electrically isolate tissue believed to be the source of an arrhythmia. During the course of such a procedure, a physician may perform, for example, radio-frequency (RF) ablation with an RF generator and a medical device such as a catheter having at least one ablation electrode. RF ablation systems may have one or more modes of operation, including for example: (i) bipolar ablation between at least two electrodes on an ablation device within a patient's body, (ii) monopolar ablation between an electrode on an ablation device within a patient's body and an external electrode contacting a patient's skin, and (iii) a combination of the monopolar and bipolar modes. The external electrode may have the form of one or more adhesive patches, which may be attached to the patient's back, and may be called a “patient return electrode.”

For acceptable performance and operation in the monopolar mode or in any combination monopolar and bipolar mode, the patient return electrode should have good contact with the patient, and a continuous electrical connection to the RF generator. Accordingly, it is desirable to provide a medical device able to verify and monitor the electrical connection between an RF generator and a patient return electrode, as well as sufficient contact between a patient return electrode and the patient.

### SUMMARY OF THE INVENTION

The present invention advantageously provides a medical system for treating patients with tissue ablation, including a generator, a catheter with at least one ablation element, a patient return electrode, and a feedback system to verify and monitor the electrical connection between the generator and the patient return electrode, as well as contact of the patient return electrode with the patient.

In particular, a medical system is provided, including a catheter having an ablation element, a radiofrequency generator connected to the catheter, the generator being operable to deliver monopolar ablation energy to the ablation element, a patient return electrode connected to the generator, and a feedback system connected to the generator and the patient return electrode, the feedback system operating to continuously monitor energy in the patient return electrode, and to cease delivery of ablation energy when the patient return electrode energy is less than a preselected threshold.

A medical system is also provided, including a catheter having an ablation element, a generator connected to the catheter, the generator being operable to deliver monopolar ablation energy to the ablation element, a patient return electrode connected to the generator, a resistor connected to the patient return electrode and defining a voltage, a convertor connected to the resistor and having an output generating a signal corresponding to the voltage, and a processor connected to the generator and the output, operable to continuously monitor the signal and cease delivery of ablation energy when the signal is less than a preselected threshold.

A method of treating a patient is provided, including providing an ablation system having an ablation element and a patient return electrode, defining a threshold, placing the ablation element proximate to a treatment site, placing the patient return electrode in contact with the patient, delivering ablation energy to the ablation element, measuring energy in the patient return electrode, comparing the measured energy to the threshold, and if the measured energy is less than the threshold, generating an alert and ceasing the delivery of ablation energy.

A medical system is provided including a catheter having an ablation element, a radiofrequency generator connected to the catheter, the generator being operable to deliver monopolar ablation energy to the ablation element, one or more patient return electrodes connected to the generator, and a feedback system connected to the generator and the patient return electrode, the feedback system operating to deliver a test signal to each patient return electrode. The test signal is insufficient to ablate a patient's tissue and is delivered before ablation energy is delivered to the ablation elements. The feedback system then continuously monitors energy in the patient return electrodes during delivery of ablation energy, and ceases delivery of ablation energy when the patient return electrode energy is less than a preselected threshold.

A method of treating a patient is provided, including providing an ablation system having an ablation element and one or more patient return electrodes, defining a threshold, placing the ablation element proximate to a treatment site, placing the patient return electrode in contact with the patient, delivering a test signal to each patient return electrode, alerting the user if the returned signal from each patient electrode is below a threshold, delivering ablation energy to the ablation element, measuring energy in the patient return electrode, comparing the measured energy to the threshold, and if the measured energy is less than the threshold, generating an alert and ceasing the delivery of ablation energy.

### BRIEF DESCRIPTION OF THE DRAWINGS

A more complete understanding of the present invention, and the attendant advantages and features thereof, will be more readily understood by reference to the following detailed description when considered in conjunction with the accompanying drawings, wherein:

FIG. 1 is an illustration of a medical system in accordance with the principles of the present invention;

FIG. 2 is an illustration of an electrical device of the medical system of FIG. 1, in accordance with the principles of the present invention;

FIG. 3 is an illustration of an electrical device having additional components in accordance with the principles of the present invention;

FIG. 4 is an illustration of a medical device in accordance with the principles of the present invention;

FIG. 5 is an illustration of an additional medical device in accordance with the principles of the present invention;

FIG. 6 is a perspective illustration of a treatment assembly for the medical device of FIG. 5, in accordance with the principles of the present invention;

FIG. 7 is an illustration of the treatment assembly of FIG. 6, in accordance with the principles of the present invention;

FIG. 8 is an illustration of another treatment assembly in accordance with the principles of the present invention;

FIG. 9 is an illustration of an additional treatment assembly in accordance with the principles of the present invention;

FIG. 10 is an illustration of a medical system having an additional patient return electrode, in accordance with the principles of the present invention;

FIG. 11 is an illustration of a medical system in accordance with the principles of the present invention, showing partial anatomical reference of a patient's heart; and

FIG. 12 is an illustration of a flow diagram in accordance with the principles of the present invention.

FIG. 13 is an illustration of a circuit diagram for detecting the presence of a patient return electrode in accordance with the principles of the present invention.

FIG. 14 is an illustration of a block diagram of a medical system in accordance with the principles of the present invention.

FIG. 15 is an illustration of a flow diagram in accordance with the principles of the present invention.

FIG. 16 is an illustration of a flow diagram in accordance with the principles of the present invention.

### DETAILED DESCRIPTION OF THE INVENTION

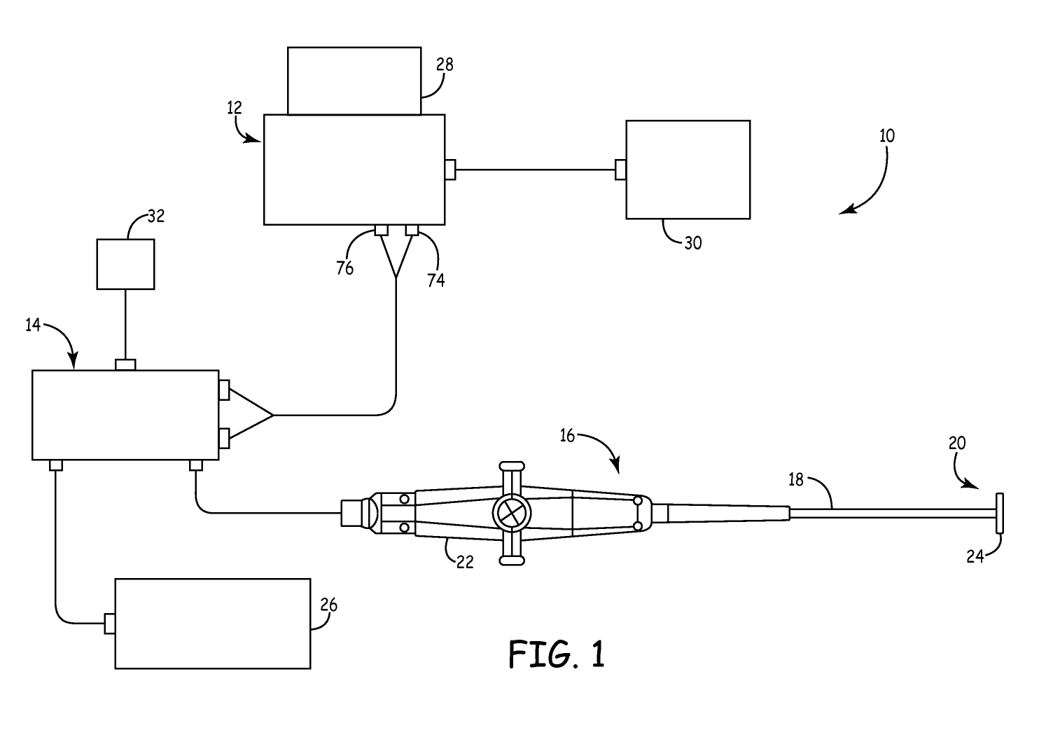

The present invention advantageously provides a medical system and method for treating patients by performing an ablation procedure in which electrical connections between portions of the medical system and the patient are verified and monitored, thereby enhancing the safety and efficacy of the therapeutic procedure. In particular and as shown in FIG. 1, an ablation therapy system, generally designated at 10, is provided for treating unwanted tissue conditions, including for example atrial fibrillation or other arrhythmias. The ablation therapy system 10 may generally include an electrical generator such as for example a radio-frequency (“RF”) generator 12, an electrocardiogram (“ECG”) interface unit 14 operably coupled to the RF generator 12, and a medical device 16. The medical device 16 may include a catheter for performing various medical treatments, including for example an electrophysiology catheter which may be operably coupled to the RF generator 12 and the ECG interface unit 14. The medical device 16 may have a shape and dimensions to reach various treatments sites, such as intraluminal access to vascular anatomy, including for example transseptal access to the left atrium of a patient's heart for subsequent treatment or ablation. The medical device 16 may generally define an elongated, flexible catheter body 18 having a distal treatment assembly 20, as well as a handle assembly 22 at or near a proximal end of the catheter body 18.

The distal treatment assembly 20 may, for example, include one or more ablation elements such as electrodes 24, each of which may be electrically coupled to the RF signal generator 12. A patient return electrode 26 may also be provided, and may include a conductive pad having a greater surface area than the electrodes 24. The patient return electrode 26 may be external to the patient, for example in contact with the patient's skin through an adhesive attachment to the back of the patient, and may be operably coupled to the ECG interface unit 14 and/or directly to the RF generator 12.

The ablation therapy system 10 may have one or more modes of operation, including for example: (i) bipolar ablation between at least two of the electrodes 24 on the medical device 16 within a patient's body, (ii) monopolar ablation between one of the electrodes 24 on the medical device 16 within a patient's body and the patient return electrode 26 contacting a patient's skin, and (iii) a combination of the monopolar and bipolar modes.

In other words, the RF generator 12 may be operable (i) to deliver ablation energy to the electrodes of the treatment assembly in a bipolar mode, directing energy between pairs of the electrodes 24 on the medical device 16, and (ii) to deliver ablation energy to one electrode 24 of the distal treatment assembly 20 and through the patient return electrode 26 in a monopolar mode. The RF generator 12 may also be operable to deliver ablation energy in a combination of the monopolar mode and the bipolar mode.

The RF generator 12 may also include a user interface 28 which may include a display and/or a remote control 30, which enable a user to select parameters for desired mapping and/or ablation treatment. The user interface 28 may allow the user to select an energy delivery mode for treatment, such as for example, selection among the delivery of only monopolar energy, only bipolar energy, or a combination of the two. When in combination mode, the user interface 28 may also allow selection a power ratio of monopolar energy to bipolar energy, such as 1:1, 2:1, or 4:1. The RF generator 12 may offer a set of specific energy ratios by default, such that the user can select one of the established energy ratios, and/or the user interface can allow the user to enter a different custom energy ratio. The user interface 28 may also allow changing the energy mode when the catheter is changed, or when the medical device 16 is moved to a different location to ablate different tissue.

The ECG interface unit 14 may also have an ECG monitoring unit or display 32 to monitor and map signals detected by the electrodes 24 of the distal treatment assembly 20 of the medical device 16. The RF generator 12 and the ECG interface unit 14 may both be operably coupled to the medical device 16. The ECG interface unit 14 may be designed to electrically isolate itself and the display 32 from the signals generated by the RF generator 12, which may include isolation from large magnitude signals and electrical noise that may result from the RF generator 12.

A feedback system 34 may be operably coupled with the RF generator 12 and the patient return electrode 26 to continuously monitor energy in the patient return electrode 26, and to cease delivery of ablation energy from the RF generator 12 if energy in the patient return electrode 26 falls below a preselected threshold. During delivery of monopolar ablation energy between one or more ablation electrodes 24 and the patient return electrode 26, in the monopolar mode or any combination mode, it is desirable to maintain good electrical contact between the RF generator 12 and the patient return electrode 26, and likewise between the patient return electrode 26 and the patient.

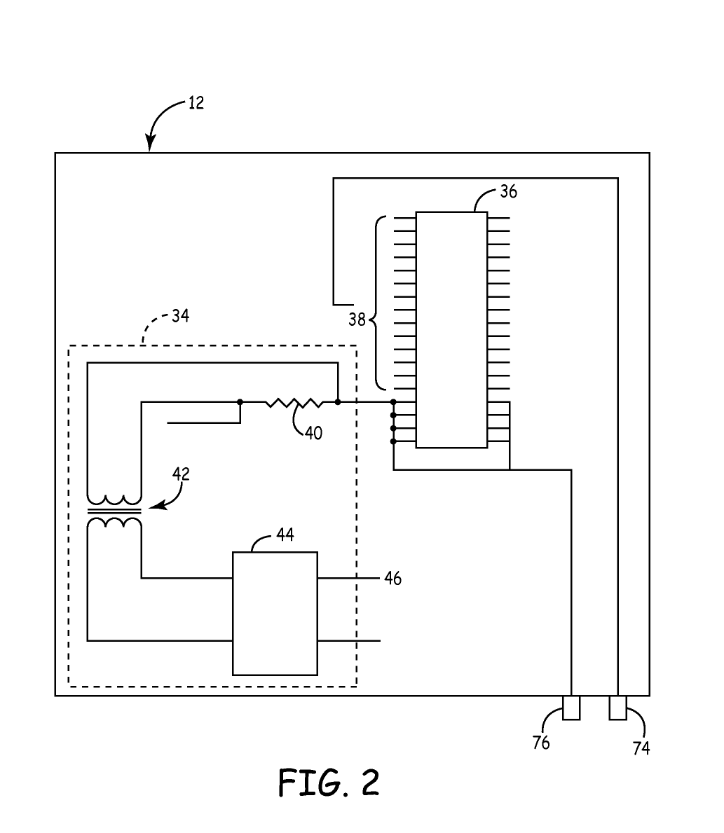

Referring to FIG. 2, the RF generator 12 may also have a processor 36 with a plurality of ablation outputs 38 coupled with an ablation connector 74. The feedback system 34 may be positioned within an enclosure of the RF generator 12 and coupled with a patient return electrode connector 76. In the generator shown in FIG. 2, the feedback system 34 is coupled in parallel with the patient return electrode 26, and may include a resistor 40, a transformer 42, and a convertor 44 operable to continuously monitor voltage across the resistor 40 and produce an output feedback signal at a convertor output 46. The resistor 40 may have a relatively small resistance, on the order of for example 0.1 ohms The transformer 42 may be connected between the resistor 40 and the convertor 44, and may have a 1:1 input to output characteristic, to isolate the circuits on either side of the transformer 42.

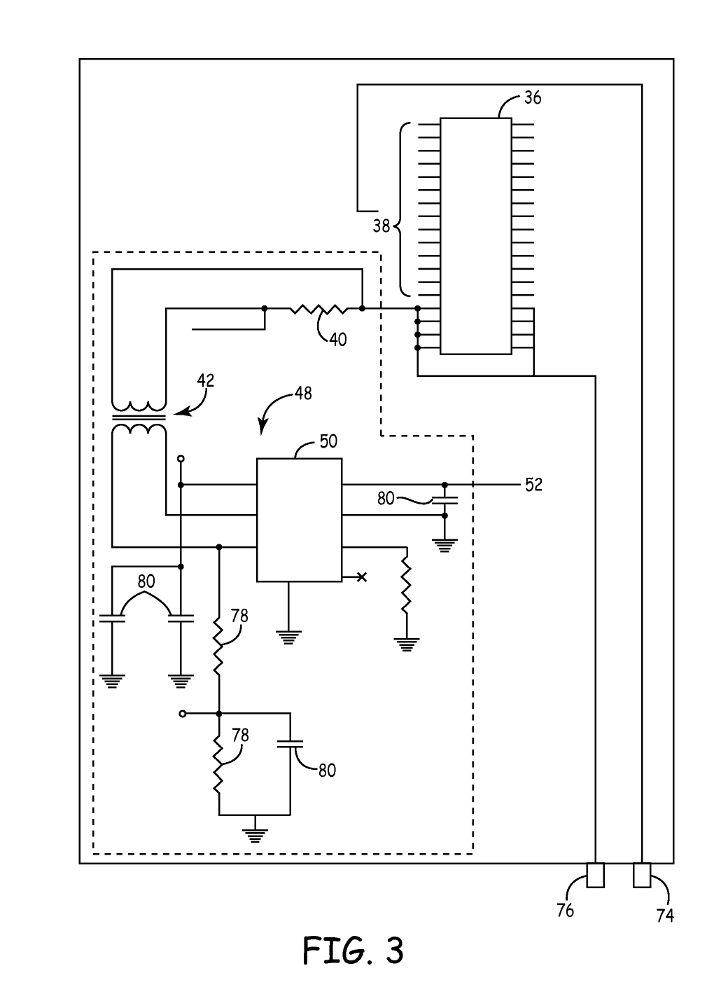

FIG. 3 depicts a more specific example of an RF generator having a more detailed feedback system. The processor 36, ablation outputs 38, resistor 40, transformer 42, ablation connector 74 and patient return electrode connector 76 are the same as those in FIG. 2. A convertor 48 is a root-mean-square (“RMS”) to direct-current (“DC”) convertor, which incorporates an integrated circuit 50 such as for example the commercially available integrated circuit convertor LTC 1968, with several components such as for example resistors 78 and capacitors 80, to produce a DC output feedback signal at a convertor output 52.

During delivery of ablation energy, the ablation therapy system 10 determines an acceptable threshold or range of an output of the feedback system 34. When the measured output is outside that acceptable threshold or range, then the ablation therapy system 10 stops ablating and may generate an alert or indication.

For example, the feedback system 34 determines a measured parameter such as a voltage at the convertor output 46 or 52. The processor 36 is operable to calculate a preselected threshold and compare it to the measured parameter. If the measured parameter at the convertor output 46 is greater than the preselected threshold, then ablation may continue as under normal operating conditions to treat the patient. If the measured parameter at the convertor output 46 is less than the preselected threshold, the feedback system 34 is operable to cease delivery of ablation energy, and may cause an alarm or warning.

The processor 36 may be programmed to calculate an expected feedback parameter, which may vary depending on the current mode of operation, and then compare the measured parameter to the expected parameter. The processor may further be programmed to calculate a ratio of the measured parameter divided by the expected parameter, and compare it to a preselected threshold. For example, the preselected threshold may be selected at any suitable amount, ranging from 100% to a small percentage. In one particular example, the threshold may be selected to equal approximately 25%.

As a specific example, the threshold value may vary according to the current mode of operation, along the lines of the following table:

| | DC output | DC output |
|---|---|---|
| Mode | All channels on | Any one channel on |
| Monopolar (1:0) | 0.25 | 0.044 |
| Combination (1:1) | 0.16 | 0.033 |
| Combination (2:1) | 0.1 | 0.025 |
| Combination (4:1) | 0.03 | 0.006 |

When the ratio of the measured parameter divided by the expected parameter is less than the threshold value, the processor may be programmed to provide an alarm and switch off the ablation energy. The alarm or warning may be auditory, visual, or tactile. After the practitioner verifies proper setup and operation of the system, an override switch (not shown) may be provided to manually reset the ablation therapy system and resume treatment of the patient.

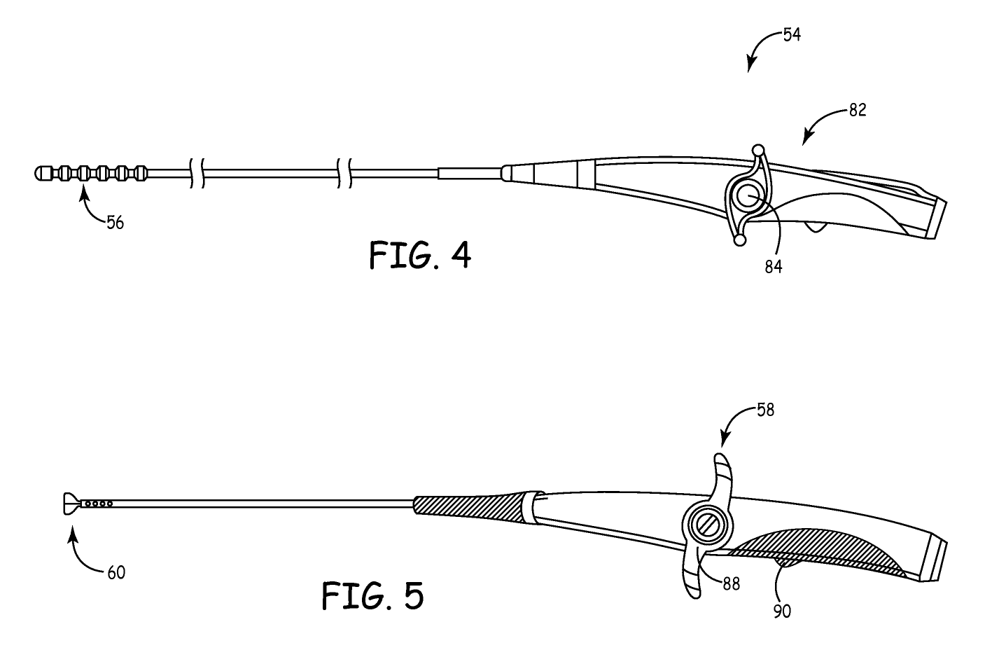

Now referring to FIGS. 4-9, some exemplary medical devices are depicted. In particular,

FIG. 4 shows an ablation catheter 54 having a distal treatment assembly 56 in which the electrodes have a linear configuration. The distal treatment assembly 56 may be used for bipolar ablation between the electrodes of the distal treatment assembly 56, or for monopolar ablation between one electrode and a patient return electrode 26, or a combination of bipolar ablation and monopolar ablation. A proximal handle 82 has a rotational actuator 84 for manipulating, bending, steering and/or reshaping the distal treatment assembly 56 into various desired shapes, curves, etc.

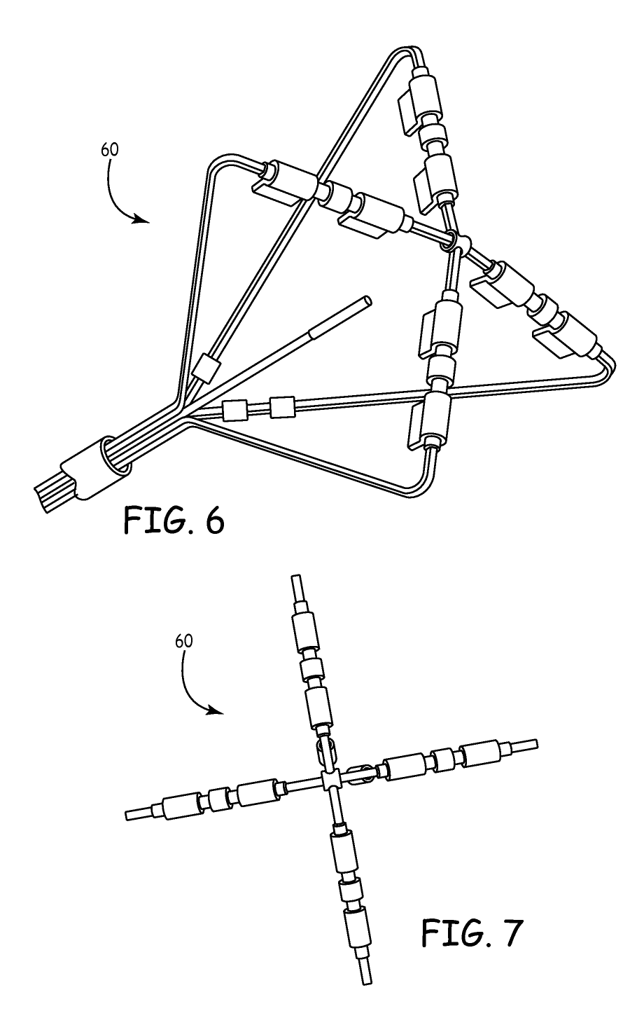

FIGS. 5-7 show an ablation catheter 58 with a distal treatment assembly 60 in which the electrodes have a planar configuration. Similar to the ablation catheter 54, the distal treatment assembly 58 may be used for bipolar ablation, monopolar ablation, or a combination thereof. A proximal handle 86 has a rotational actuator 88 for manipulating a distal portion of the ablation catheter 58, and a linear actuator 90. The linear actuator 90 can advance the distal treatment assembly 60 distally beyond a catheter shaft, and retract the distal treatment assembly 60 proximally inside the catheter shaft. When the distal treatment assembly 60 is advanced distally, it may resiliently expand from a compressed arrangement inside the catheter shaft to the deployed arrangement shown in FIGS. 6 and 7.

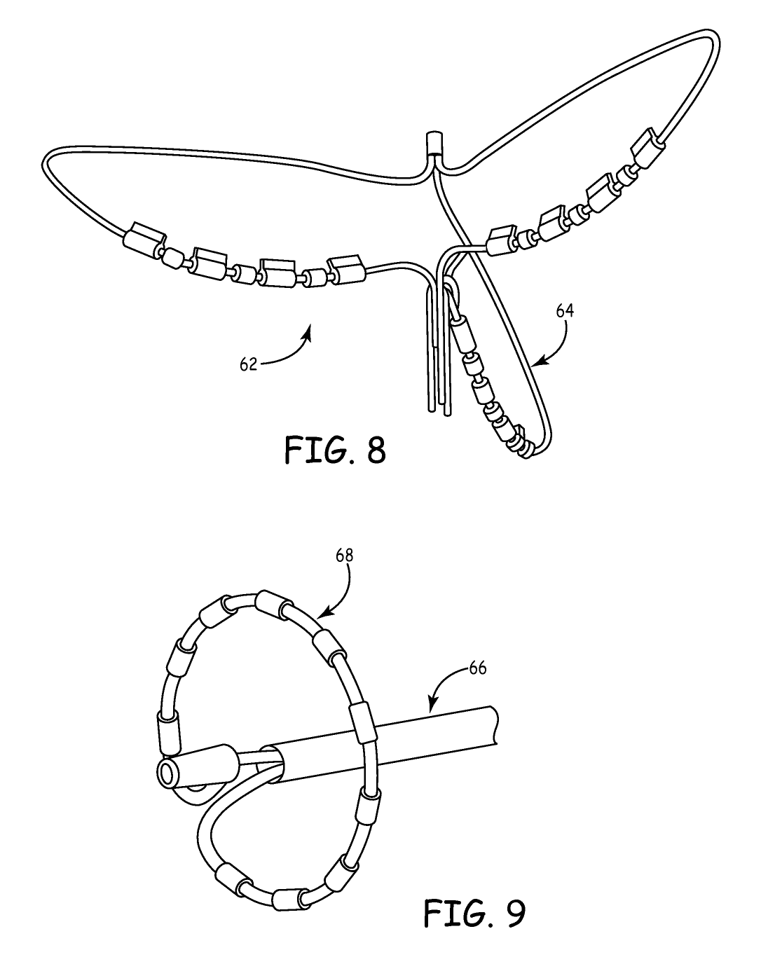

FIG. 8 shows a catheter 62 which has a distal treatment assembly 64 having a resilient framework in which the electrodes have a proximally-directed configuration, which may for example be used for transseptal treatments of a patient's heart.

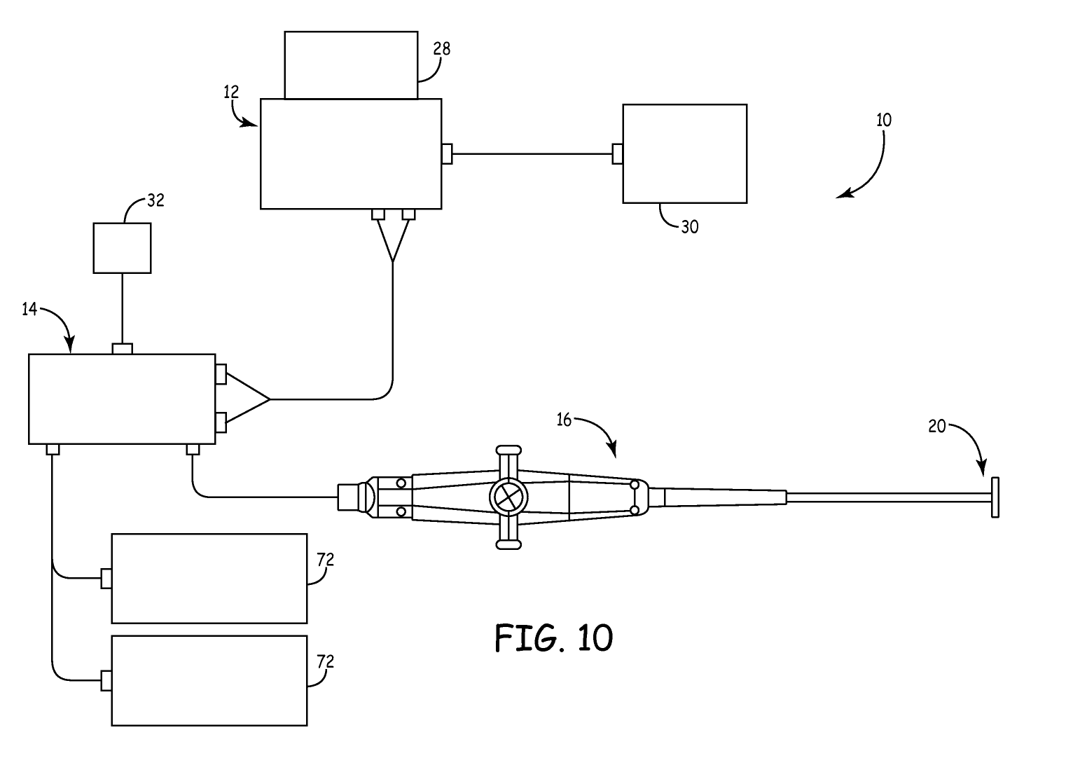

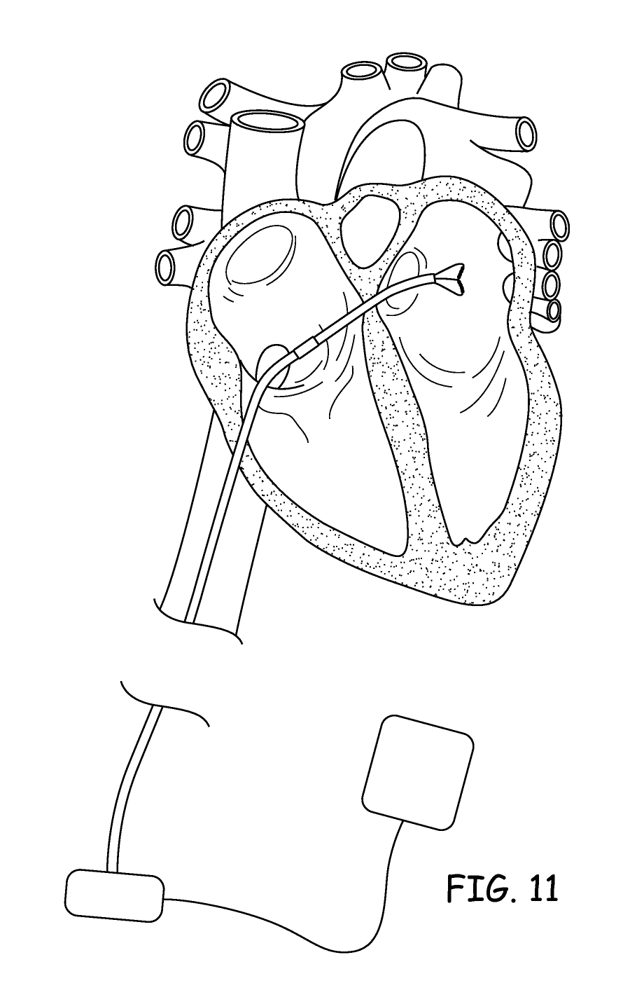

FIG. 9 shows a catheter 66 which has a distal treatment assembly 68 in which the electrodes have an adjustable linear, planar, or spiral configuration. Now referring to FIG. 10, an ablation treatment system 70 may also a plurality of patient return electrodes 72, with a processor operable to independently monitor electrical energy in each patient return electrode 72. The processor may also operate to calculate a combined expected feedback parameter from both patient return electrodes 72, determine a combined measured parameter for both patient return electrodes 72, calculate a ratio of the combined measured parameter divided by the combined expected parameter, and compare the ratio to a preselected threshold.

Accordingly, the medical device 16 may be used to investigate and treat aberrant electrical impulses or signals in a selected tissue region, such as in the heart. Primarily, the distal treatment assembly 20 may be advanced through the patient's vasculature via the femoral artery over a previously inserted guidewire. The distal treatment assembly 20 may then be advanced into the right atrium and into proximity of a pulmonary vein, for example.

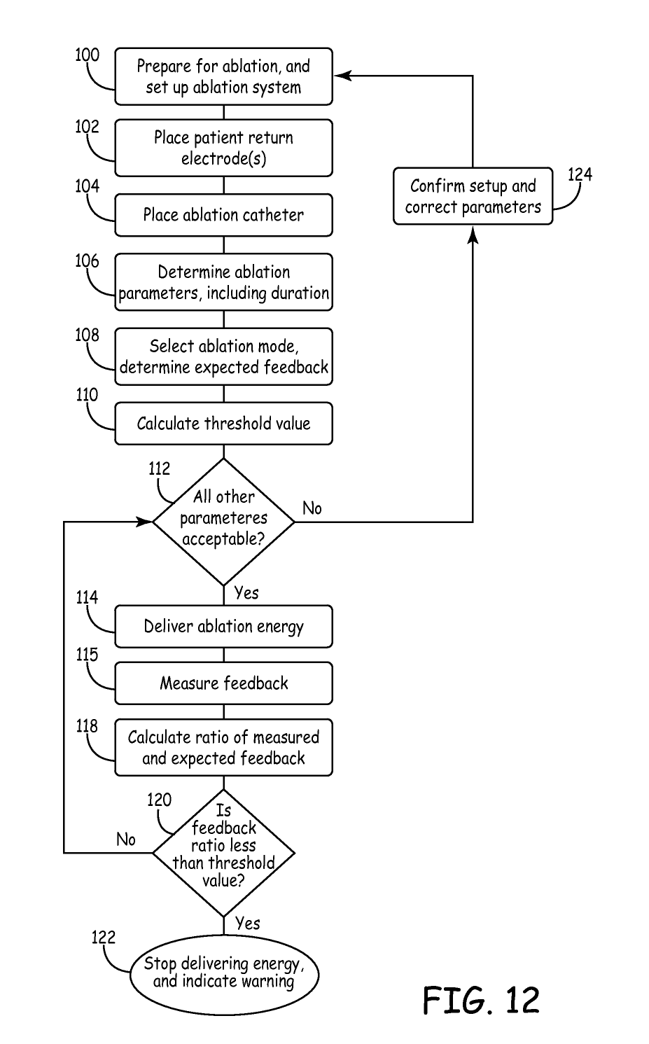

In an exemplary use of the present system as illustrated in the flow diagram of FIG. 12, the medical system is first prepared for ablation, and the ablation system is set up (step 100). One or more patient return electrodes are placed (step 102), and the ablation catheter is placed so that the distal treatment assembly 20 is in the desired position for treatment (step 104). Various ablation parameters are determined, including the intended duration of ablation (step 106). The desired ablation mode is selected, for example monopolar ablation, bipolar ablation, or a specific combination thereof, and the expected feedback is determined (step 108). A threshold value is calculated based on the selected parameters and mode of ablation (step 110), which may be a preselected percentage of the expected feedback. If all parameters are not acceptable (step 112), then the setup and parameters are evaluated and corrected (step 124). If all parameters are acceptable (step 112), then delivery of ablation energy may commence (step 114).

During ablation, the feedback signal or output is continuously verified and monitored (step 116). A ratio of the measured feedback divided by the expected feedback is calculated (step 118). If the feedback ratio is greater than the threshold value (step 120), then all parameters continue to be evaluated (step 112), and ablation continues as under normal conditions until a parameter is not acceptable or the intended ablation duration completes. If the feedback ratio is equal to or less than the threshold value (step 120), then ablation stops, and the system indicates a warning (step 122).

Sufficient contact with tissue may be determined through fluoroscopic imaging. In addition, the location and tissue contact can be confirmed using the electrodes 24 of the medical device. For example, an electrophysiologist can map the contacted tissue to not only determine whether or not to ablate any tissue, but to also confirm tissue contact which is identified in the mapping procedure. If conditions are determined to be inadequate, an operator may adjust the shape of carrier assembly, and/or the operator may reposition the distal treatment assembly 20 against tissue through various manipulations performed at the proximal end of the medical device 16. Moreover, it will be appreciated that other conventional mapping catheters can be applied to map signals, such as a standard electrophysiology lasso catheter.

Once sufficient tissue contact has been established and the mapping procedure has confirmed the presence of aberrant conductive pathways, ablation energy may be passed through the electrodes 24 (for example, 5-10 Watts) of the distal treatment assembly 20. The distal treatment assembly 20 and the RF signal generator 12 may cooperate to deliver RF energy in monopolar, bipolar or combination monopolar-bipolar energy delivery modes, simultaneously or sequentially, and with or without durations of terminated energy delivery.

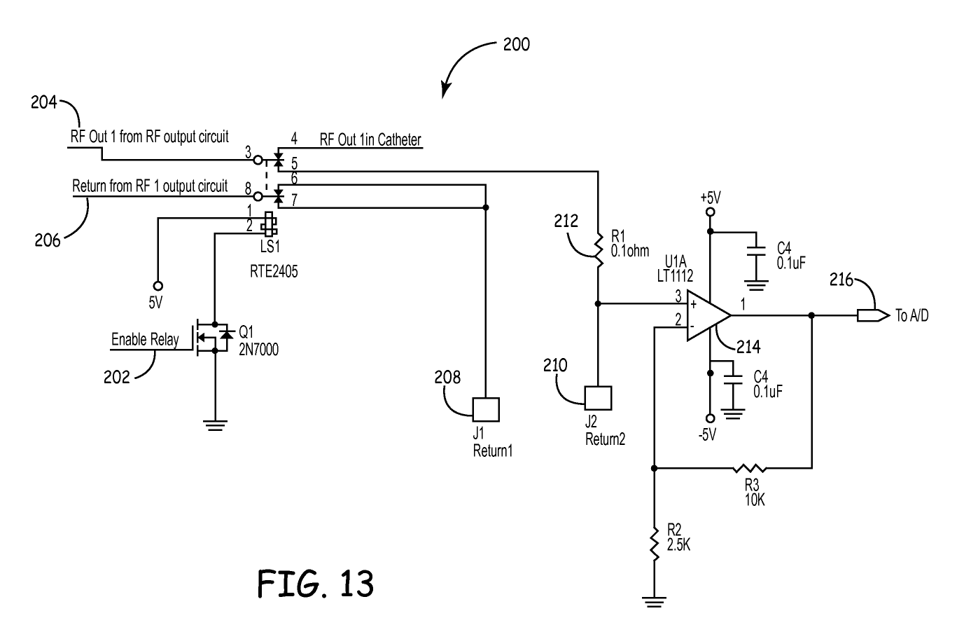

Referring now to FIG. 13, a return test circuit 200 is illustrated. The circuit shown is for use in a medical system that has two return pads connected to a patient. In this embodiment, as shown in FIG. 13, each return pad has a dedicated return path, designated as first return path 208 and second return path 210. In the circuit of FIG. 13, activation of enable relay 200 causes RF output 204 to connect to first return path 208 and return conductor 206 to connect to second return path 210.

A small duty cycle RF signal is generated from RF output 204 such that the current through the patient's tissue does not exceed 150 mA. This RF output is insufficient to ablate the patient's tissue. The frequency of the signal may be at that used to perform an RF ablation, such as between 400 kHz to 600 kHz. Alternatively, the frequency may be at some other frequency. Any frequency that is sufficiently high to avoid stimulating cardiac tissue may be used, such as 5 kHz or greater. If the return pads are securely connected to the patient's body, the current flows through RF output 204, the current sense resistor 212, second return path 210, through the patient's tissue (not shown), first return path 208, and back to the return conductor 206.

The current is measured by amplifier 214 and transmitted to an analog-to-digital (A/D) converter through output 216. The value measured by the A/D converter is compared to threshold values in the manner described previously to determine whether or not the return pads are securely connected to the patient's body.

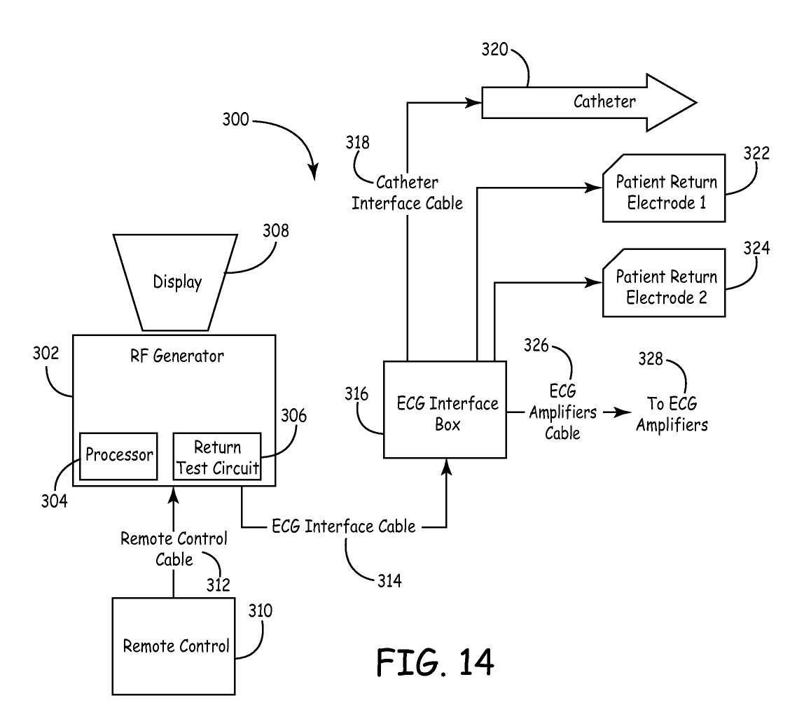

Referring now to FIG. 14, a block diagram of an embodiment of the present invention is shown. Ablation system 300 includes RF generator 302. Processor 304 is programmed to control RF generator 302 to deliver controlled radiofrequency (RF) energy to user selectable electrodes on cardiac ablation catheter 302. Ablation catheter 302 may include a multi-electrode catheter such as those depicted in FIGS. 3-7. Alternatively, catheter 302 may have only a single electrode, often referred to as a tip or focal catheter. The RF generator 302 includes return test circuit 306 as described in FIG. 13. Display 308 may be a standard personal computer monitor, such as a flat-panel monitor.

Remote control 310 provides full control of the RF generator 302 and can be placed within the electro-physiologists reach or EP lab control room. The remote control can be covered by a sterile barrier when placed near or within the sterile field. It is connected to the Generator with the detachable remote control cable 312. ECG interface box 316 interfaces the catheter 320 with the Electrophysiology (EP) laboratory's ECG amplifier systems for collection of intracardiac electrograms through ECG amplifiers cable 326. Element 328 depicts the transmission of the signal to the EP laboratory's ECG amplifier system. ECG interface box 316 also connects the catheter 320, patent return electrode 1 (322) and patient return electrode 2 (324) to the RF generator 302 through ECG interface cable 314.

Patient Return Electrodes attach to the patient's skin and provide a return path for the unipolar RF energy from the electrodes on catheter 320. Multiple patient return electrodes may be used. In the embodiment depicted in FIG. 14, two return electrodes are applied to the patient, designated as patient return electrode 1 (322) and patient return electrode 2 (324).

In operation, when return test circuit 306 is activated, a test signal as described in connection with FIG. 13 is applied to each patient return electrode. A return signal from each patient return electrode is measured at by a processor (not shown) in the RF generator 302.

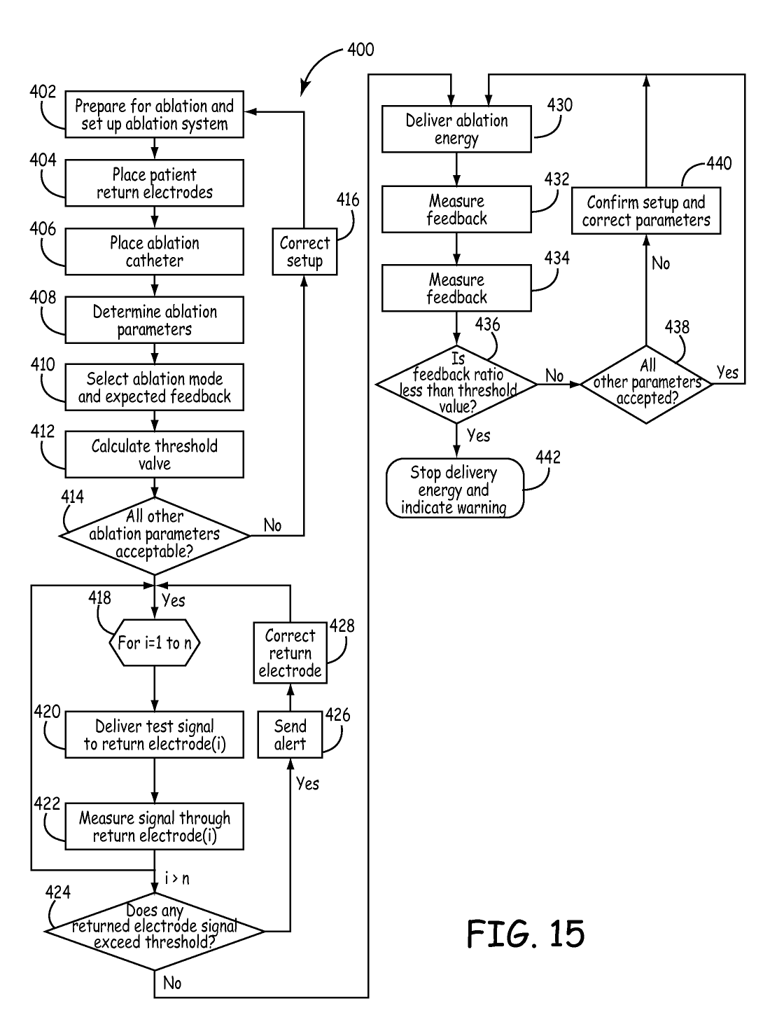

Referring now to FIG. 15, an exemplary use of the present system that incorporates the return test circuit 200 of FIG. 13 is illustrated in flow diagram 400. In an exemplary use of the present system as illustrated in the flow diagram of FIG. 15, the medical system is first prepared for ablation, and the ablation system is set up (step 402). One or more patient return electrodes are placed (step 404), and the ablation catheter is placed so that the distal treatment assembly 20 is in the desired position for treatment (step 406). Various ablation parameters are determined, including the intended duration of ablation (step 408). The desired ablation mode is selected, for example monopolar ablation, bipolar ablation, or a specific combination thereof, and the expected feedback is determined (step 410). The ablation parameters can include which channels are to be used to deliver ablation energy, and which channels are unused. A threshold value is calculated based on the selected parameters and mode of ablation (step 412), which may be a preselected percentage of the expected feedback. If all parameters are not acceptable (step 414), then the setup and parameters are evaluated and corrected (step 416). If all parameters are acceptable (step 414), then, prior to the delivery of ablation energy or ablation therapy, the placement of each of a plurality of patient return electrodes is evaluated (step 418).

Step 414 implements a decision loop for each return electrode from 1 to n, where n is the number of return electrode. For each electrode, a test signal, which may be the low-duty cycle RF signal as described in connection with FIG. 13, is delivered to the return electrode. A signal is measured through the return electrode (step 422). After a test signal has been delivered to and measured through each electrode, it is determined whether any returned signal exceeds a threshold (step 424). Exceeding the threshold indicates that the return electrode is mispositioned on the patient's body.

If step 424 returns a yes, indicating that at least one returned signal exceeds a threshold, then the system sends an alert to the operator (step 428). The positioning of the return electrodes is corrected (step 428) and the loop initiated by step 418 re-executes.

If step 424 returns a no, indicating that each return electrode is properly positioned, then the system proceeds to deliver ablation energy to the patient (step 430).

During ablation, the feedback signal or output is continuously verified and monitored (step 432). A ratio of the measured feedback divided by the expected feedback is calculated (step 434). If the feedback ratio is greater than the threshold value (step 436), then all parameters continue to be evaluated (step 438), and ablation continues as under normal conditions until a parameter is not acceptable or the intended ablation duration completes. If any other parameter is out of the acceptable range, then the user confirms the setup and corrects the unacceptable parameters (step 440). If the feedback ratio is equal to or less than the threshold value (step 436), then ablation stops, and the system indicates a warning (step 442).

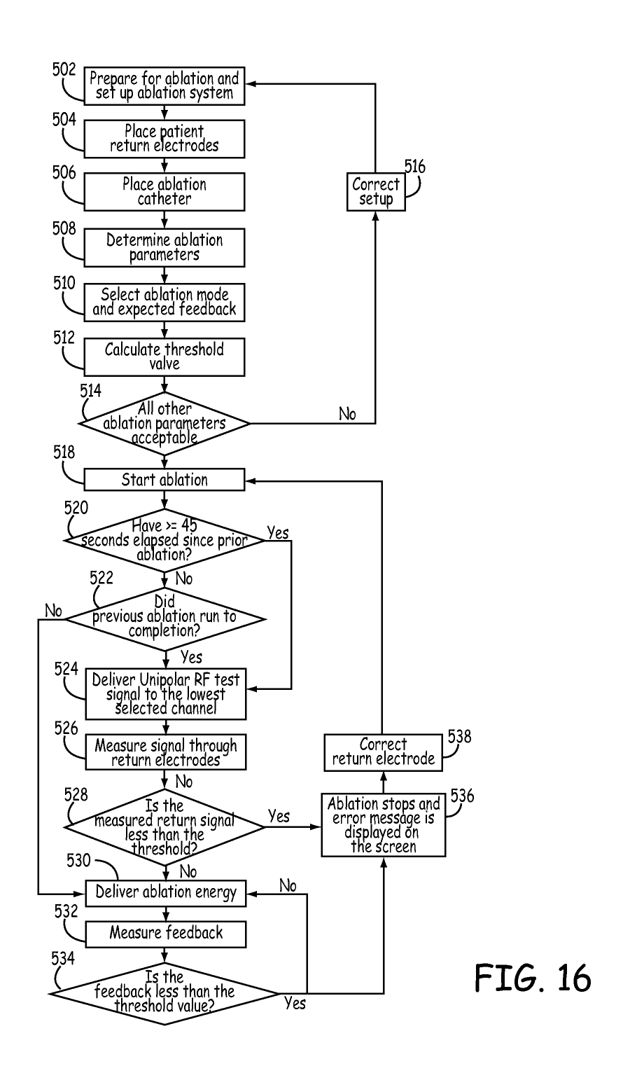

Referring now to FIG. 16, another exemplary use of the present system that incorporates the return test circuit 200 of FIG. 13 is illustrated in flow diagram 500. In an exemplary use of the present system as illustrated in the flow diagram of FIG. 16, the medical system is first prepared for ablation, and the ablation system is set up (step 502). One or more patient return electrodes are placed (step 504), and the ablation catheter is placed so that the distal treatment assembly 20 is in the desired position for treatment (step 506). Various ablation parameters are determined, including the intended duration of ablation (step 508). The desired ablation mode is selected, for example monopolar ablation, bipolar ablation, or a specific combination thereof, and the expected feedback is determined (step 510). The ablation parameters can include which channels are to be used to deliver ablation energy, and which channels are unused. A threshold value is calculated based on the selected parameters and mode of ablation (step 512), which may be a preselected percentage of the expected feedback. If all parameters are not acceptable (step 514), then the setup and parameters are evaluated and corrected (step 516). If all parameters are acceptable (step 514), then the ablation begins in step 518.

Step 520 determines if more than 45 seconds have elapsed since the previous ablation. If yes, then the procedure jumps to step 524. If no, the procedure moves to step 522, in which it is determined whether the previous ablation has run to completion, or whether it was terminated prior to completion for any reason. If yes, the procedure moves to step 524.

In step 524, a test signal is delivered to the lowest numbered channel that has been selected for delivery of ablation energy. For this selected channel, a test signal, which may be the low-duty cycle RF signal as described in connection with FIG. 13, is delivered to the return electrode. A signal is measured through the return electrode (step 526). After a test signal has been delivered to and measured through each electrode, it is determined whether any returned signal is less than the threshold (step 528). Exceeding the threshold indicates that the return electrode is positioned correctly on the patient's body.

If step 528 returns a yes, indicating that at least one returned signal is less than the threshold, then the system sends an alert to the operator (step 526). The positioning of the return electrodes is corrected (step 528) and the loop initiated by step 518 re-executes.

If step 528 returns a no, indicating that each return electrode is properly positioned, then the system proceeds to deliver ablation energy to the patient (step 530).

During ablation, the feedback signal or output is continuously verified and monitored (step 532). In step 532, the measured return current (feedback) is compared to the threshold value. If the feedback is greater than the threshold value (step 534), then all parameters continue to be evaluated (step 524), and ablation continues as under normal conditions until a parameter is not acceptable or the intended ablation duration completes. If the feedback is equal to or less than the threshold value (step 534), then ablation stops, and the system indicates a warning (step 536). The user is directed to correct the return electrode (538) and the procedure resumes at step 518.

Sufficient contact with tissue may be determined through fluoroscopic imaging. In addition, the location and tissue contact can be confirmed using the electrodes 24 of the medical device. For example, an electrophysiologist can map the contacted tissue to not only determine whether or not to ablate any tissue, but to also confirm tissue contact which is identified in the mapping procedure. If conditions are determined to be inadequate, an operator may adjust the shape of carrier assembly, and/or the operator may reposition the distal treatment assembly 20 against tissue through various manipulations performed at the proximal end of the medical device 16. Moreover, it will be appreciated that other conventional mapping catheters can be applied to map signals, such as a standard electrophysiology lasso catheter.

Once sufficient tissue contact has been established and the mapping procedure has confirmed the presence of aberrant conductive pathways, ablation energy may be passed through the electrodes 24 (for example, 5-10 Watts) of the distal treatment assembly 20. The distal treatment assembly 20 and the RF signal generator 12 may cooperate to deliver RF energy in monopolar, bipolar or combination monopolar-bipolar energy delivery modes, simultaneously or sequentially, and with or without durations of terminated energy delivery.

While examples and illustrations of particular medical system configurations have been provided, it is understood that various arrangements, shapes, configurations, and/or dimensions may be included in the medical device of the present invention, including but not limited to those illustrated and described herein. Also, though monopolar and bipolar RF ablation energy may be the selected forms of energy to pass through the electrodes of the medical device, other forms of ablation energy may be additionally or alternatively emitted from the treatment assembly, including electrical energy, magnetic energy, microwave energy, thermal energy (including heat and cryogenic energy) and combinations thereof. Moreover, other forms of energy that may be applied can include acoustic energy, sound energy, chemical energy, photonic energy, mechanical energy, physical energy, radiation energy and a combination thereof.

It should be understood that an unlimited number of configurations for the present invention could be realized. The foregoing discussion describes merely exemplary embodiments illustrating the principles of the present invention, the scope of which is recited in the following claims. In addition, unless otherwise stated, all of the accompanying drawings are not to scale. Those skilled in the art will readily recognize from the description, claims, and drawings that numerous changes and modifications can be made without departing from the spirit and scope of the invention.

1. A medical system, comprising:
a catheter having an ablation element;
a radiofrequency generator connected to the catheter;
a patient return electrode connected to the generator;
wherein the generator is operable to deliver radiofrequency ablation energy to the ablation element and to deliver a radiofrequency test signal to the ablation element, wherein the power delivered by said test signal is insufficient to ablate tissue; and
a feedback system connected to the generator and the patient return electrode, the feedback system operating to measure a signal from the patient return electrode when the generator is delivering the radiofrequency test signal.

2. The medical system according to claim 1, further comprising a processor connected to the generator and the feedback system, wherein the processor is operable to calculate an expected value of the signal from the patient return electrode and compare it to the measured signal from the patient return electrode.

3. The medical system according to claim 2, wherein the processor is programmed to calculate a ratio of the measured signal from the patient return electrode divided by the expected value of the signal from the patient return electrode, and compare it to a preselected threshold.

4. The medical system according to claim 3, wherein the preselected threshold equals approximately 25%.

5. The medical system according to claim 1, further comprising a second patient return electrode and a processor, the processor being operable to independently monitor a signal returned from each patient return electrode.

6. The medical system according to claim 1, further comprising a second patient return electrode and a processor, wherein the processor is operable to calculate a combined expected feedback parameter, determine a combined measured parameter for both patient return electrodes, calculate a ratio of the combined measured parameter divided by the combined expected parameter, and compare the ratio to the preselected threshold.

7. The medical system of claim 1, wherein the feedback system further operates to continuously monitor a signal from the patient return electrode while ablation therapy is delivered, and to cease delivery of ablation energy when the patient return electrode signal is less than a preselected threshold.

8. The medical system of claim 1, wherein the radiofrequency test signal has a frequency greater than or equal to 5 kHz.

9. The medical system of claim 1, wherein the catheter has a plurality of ablation elements.

10. A method of treating a patient, comprising:
providing an ablation system having an ablation element and a patient return electrode;
defining a threshold;
placing the ablation element proximate to a treatment site;
placing the patient return electrode in contact with the patient;
delivering a test signal to the ablation element;
measuring a signal from the patient return electrode;
comparing the measured signal to the threshold; and
if the measured signal is less than the threshold, generating an alert.

11. The method of claim 10, wherein the measured signal in the patient return electrode is the current through the return electrode, and further comprising determining an expected current in the patient return electrode during delivery of the test signal to the ablation element, wherein the threshold is calculated as a percentage of the expected power.

12. The method of claim 10, wherein the threshold is about 25% of the expected current.

13. The method of claim 10, further comprising the step of delivering ablation energy to the ablation element, wherein the step of delivering a test signal to the ablation element is performed before the step of delivering ablation energy.

14. The method of claim 13, further comprising the step of continuously measuring energy in the patient return electrode while delivering ablation energy to the ablation element.

15. The method of claim 10, wherein the test signal has a frequency greater than or equal to 5 kHz.
